(19) United States
(12) Patent Application Publication
(10) Pub. No.: US 2023/0233752 A1
(43) Pub. Date: Jul. 27, 2023

(54) SMART INJECTOR TURN KNOBS

(71) Applicant: BAYER HEALTHCARE LLC, WHIPPANY, NY (US)

(72) Inventors: LINDA VAN ROOSMALEN, GIBSONIA, PA (US); CHRISTOPHER SCUTT, MURRYSVILLE, PA (US)

(21) Appl. No.: 17/998,294

(22) PCT Filed: May 19, 2021
(86) PCT No.: PCT/US2021/033056

§ 371 Date: Nov. 9, 2022

Related U.S. Application Data
(60) Provisional application No. 62/704,628, filed on May 19, 2020.

(51) Int. Cl. A61M 5/00; A61M 5/145; A61M 5/148

(52) U.S. Cl. CPC A61M 5/007; A61M 5/148; A61M 5/1452; A61M 2005/14208; A61M 2005/14506

(57) ABSTRACT

A fluid injector system (1000) is configured to perform an injection protocol. The fluid injector system includes a housing (11) and a controller (900) operatively associated with a user input device (40) and a fluid actuator (16). The controller includes at least one processor programmed or configured to determine an orientation of the housing, receive at least one signal from the user input device, determine a direction of fluid actuation based on the orientation of the housing and the at least one signal, and actuate the fluid actuator in the direction of fluid actuation. The direction of fluid actuation corresponds to at least one of actuating the fluid actuator to inject fluid from a fluid reservoir and actuating the fluid actuator to draw fluid into the fluid reservoir.

### CROSS-REFERENCE TO RELATED APPLICATION

This application claims the benefit of U.S. Provisional Pat. Application No. 62/704,628, filed on May 19, 2020, the disclosure of which is hereby incorporated by reference in its entirety.

### BACKGROUND OF THE DISCLOSURE

### Field of the Disclosure

The present disclosure is related to a fluid injector system and, more particularly, to a fluid injector system having user input devices for controlling fluid actuator movement and other functions of the system.

### Description of Related Art

In many medical diagnostic and therapeutic procedures, a medical practitioner, such as a physician or radiologist, injects a patient with one or more fluids. In recent years, a number of injector-actuated syringes and powered injectors for pressurized injection of fluids have been developed for use in procedures such as coronary angiography (CV), computed tomography (CT), molecular imaging (such as positron emission tomography (PET) imaging), and magnetic resonance imaging (MRI). In these procedures, a fluid, such as a contrast agent, may be used to highlight or enhance certain internal organs or portions of the body during an imaging process. Meanwhile, saline, or a similar flushing agent, may be used to ensure complete injection of the bolus of the contrast agent or to adjust the concentration of the contrast agent.

Powered injectors include either one or two drive mechanisms and thus are often referred to as single-head or dual head systems, respectively. In each case, a drive mechanism typically includes a piston and a driving element (e.g. a ball screw or the like) for extending and retracting the piston within each syringe to affect fluid delivery of the contrast or saline therein. For example, the piston may be driven proximally within the barrel of the syringe to fill the syringe, and the piston may be driven distally within the barrel of the syringe to expel fluid during an injection procedure or to purge and/or prime the system. Movement of the piston during an injection procedure is typically controlled via an electronic controller (e.g. a processor). However, in some cases, it may be desirable to extend and/or retract the piston manually, e.g. during a purging operation to ensure all air has been removed from the syringe(s) and other portions of the fluid path (e.g., one or more tubing sets, administration line and associated catheter) connected thereto. For manual control, fluid injectors may have a knob in mechanical connection with the driving element of the piston (e.g. a ball screw).

Manual control knobs that are mechanically connected to the driving element have several deficiencies. First, the direction in which the control knob must be rotated to achieve the desired piston movement may change depending on the orientation of the powered injector. As such, operators may inadvertently drive the piston in an unintended direction. Second, the operator must generally be in close proximity to the powered injector, and thus may be exposed to radiation from imaging equipment, in order to actuate the control knobs. In addition to these specific deficiencies, manual control knobs may be generally cumbersome and unintuitive to operate and provide limited functionality.

### SUMMARY OF THE DISCLOSURE

In view of the foregoing, there exists a need for fluid injector systems with more intuitive and functional devices for manually controlling the pistons. Additionally, there exists a need for methods for operating such fluid injector systems and for computer program products for executing such methods. Accordingly, embodiments of the present disclosure are directed to a fluid injector system configured to perform an injection protocol. The fluid injector system includes a housing and a controller operatively associated with a user input device and a fluid actuator. The controller includes at least one processor programmed or configured to determine an orientation of the housing, receive at least one signal from the user input device, determine a direction of fluid actuation based on the orientation of the housing and the at least one signal, and actuate the fluid actuator in the direction of fluid actuation. The direction of fluid actuation corresponds to at least one of actuating the fluid actuator to inject fluid from a fluid reservoir and actuating the fluid actuator to draw fluid into the fluid reservoir.

In some embodiments, the fluid actuator is at least one of a piston actuator, and pump actuator, and a compressive actuator.

In some embodiments, the at least one processor is further programmed or configured to determine a change in the orientation of the housing and change the direction of fluid actuation in response to determining the change in the orientation of the housing.

In some embodiments, the orientation of the housing includes a degree of tilt relative to a neutral plane.

In some embodiments, the at least one signal from the user input device includes a rotation direction of the user input device.

In some embodiments, the at least one processor is further programmed or configured to determine a load on the fluid actuator and adjust a resistance of the user input device based on the load.

In some embodiments, the at least one processor is further programmed or configured to determine at least one characteristic of a fluid path set and adjust at least one parameter of the injection protocol based on the at least one characteristic of the fluid path set.

In some embodiments, the at least one characteristic of the fluid path set includes a compliance rating of the fluid path set or of the fluid reservoir.

In some embodiments, the fluid injector system further includes a scanner configured to scan a tag of the fluid path set to determine the at least one characteristic of the fluid path set.

In some embodiments, the at least one processor is further programmed or configured to determine a current status of the fluid injector system and disable at least one direction of fluid actuator movement based on the current status.

In some embodiments, the at least one processor is further programmed or configured to set a fluid actuation speed based on at least one of the at least one signal from the user input device and a current status of the fluid injector system, and actuate the fluid actuator at the fluid actuation speed.

In some embodiments, the fluid actuation speed is set proportional to a speed at which the user input device is moved.

In some embodiments, the at least one processor is further programmed or configured to receive at least one additional signal from the user input device and adjust at least one of a height and the orientation of the housing based on the at least one additional signal.

In some embodiments, the fluid injector system further includes at least one valve. The at least one processor is further programmed or configured to actuate the valve in response to determining the direction of fluid actuation.

In some embodiments, the user input device is at least one of mounted to the housing and mounted remotely from the housing.

Other embodiments of the present disclosure are directed to a computer program product for actuating a fluid actuator of a fluid injector system configured to perform an injection protocol. The computer program product includes at least one non-transitory computer-readable medium including one or more instructions that, when executed by at least one processor, cause the at least one processor to determine an orientation of a housing of the fluid injector system, receive at least one signal from a user input device of the fluid injector system, determine a direction of fluid actuation based on the orientation of the housing and the at least one signal, and actuate the fluid actuator in the direction of fluid actuation. The direction of fluid actuation corresponds to at least one of actuating the fluid actuator to inject fluid from a fluid reservoir and actuating the fluid actuator to draw fluid into the fluid reservoir.

In some embodiments, the fluid actuator is at least one of a piston actuator, and pump actuator, and a compressive actuator.

In some embodiments, the one or more instructions further cause the at least one processor to determine a change in the orientation of the housing and change the direction of fluid actuation in response to determining the change in the orientation of the housing.

In some embodiments, the orientation of the housing includes a degree of tilt relative to a neutral plane.

In some embodiments, the at least one signal from the user input device includes a rotation direction of the user input device.

In some embodiments, the one or more instructions further cause the at least one processor to determine a load on the fluid actuator and adjust a resistance of the user input device based on the load.

In some embodiments, the one or more instructions further cause the at least one processor to determine at least one characteristic of a fluid path set and adjust at least one parameter of the injection protocol based on the at least one characteristic of the fluid path set.

In some embodiments, the at least one characteristic of the fluid path set includes a compliance rating of the fluid path set or of the fluid reservoir.

In some embodiments, determining the at least one characteristic of a fluid path set includes scanning a tag of the fluid path set.

In some embodiments, the one or more instructions further cause the at least one processor to determine a current status of the fluid injector system and disable at least one direction of fluid movement based on the current status.

In some embodiments, the one or more instructions further cause the at least one processor to set a fluid actuation speed based on at least one of the at least one signal from the user input device and a current status of the fluid injector system, and actuate the fluid actuator at the actuation speed.

In some embodiments, the fluid actuation speed is set proportional to a speed at which the user input device is moved.

In some embodiments, the one or more instructions further cause the at least one processor to receive at least one additional signal from the user input device and adjust at least one of a height and the orientation of the housing based on the at least one additional signal.

In some embodiments, the one or more instructions further cause the at least one processor to actuate at least one valve of the fluid injector system in response to determining the direction of fluid actuation.

Other embodiments of the present disclosure are directed to a method for actuating a fluid actuator of a fluid injector system configured to perform an injection protocol. The method includes determining an orientation of a housing of the fluid injector system, receiving at least one signal from a user input device of the fluid injector system, determining a direction of fluid actuation based on the orientation of the housing and the at least one signal, and actuating the fluid actuator in the direction of fluid actuation. The direction of fluid actuation corresponds to at least one of actuating the fluid actuator to inject fluid from a fluid reservoir and actuating the fluid actuator to draw fluid into the fluid reservoir.

In some embodiments, the fluid actuator is at least one of a piston actuator, and pump actuator, and a compressive actuator.

In some embodiments, the method further includes determining a change in the orientation of the housing and changing the direction of fluid actuation in response to determining the change in the orientation of the housing.

In some embodiments, the orientation of the housing includes a degree of tilt relative to a neutral plane.

In some embodiments, the at least one signal from the user input device includes a rotation direction of the user input device.

In some embodiments, the method further includes determining a load on the fluid actuator and adjusting a resistance of the user input device based on the load.

In some embodiments, the method further includes determining at least one characteristic of a fluid path set and adjusting at least one parameter of the injection protocol based on the at least one characteristic of the fluid path set.

In some embodiments, the at least one characteristic of the fluid path set includes a compliance rating of the fluid path set or of the fluid reservoir.

In some embodiments, determining the at least one characteristic of a fluid path set includes scanning a tag of the fluid path set.

In some embodiments, the method further includes determining a current status of the fluid injector system and disabling at least one direction of fluid movement based on the current status.

In some embodiments, the method further includes setting a fluid actuation speed based on at least one of the at least one signal from the user input device and a current status of the fluid injector system, and actuating the fluid actuator at the actuation speed.

In some embodiments, the fluid actuation speed is set proportional to a speed at which the user input device is moved.

In some embodiments, the method further includes receiving at least one additional signal from the user input device and adjusting at least one of a height and the orientation of the housing based on the at least one additional signal.

Various other embodiments of the present disclosure are recited in one or more of the following numbered clauses:

Clause 1. A fluid injector system configured to perform an injection protocol, the fluid injector system comprising: a housing; and a controller operatively associated with a user input device and a fluid actuator, the controller comprising at least one processor programmed or configured to: determine an orientation of the housing; receive at least one signal from the user input device; determine a direction of fluid actuation based on the orientation of the housing and the at least one signal; and actuate the fluid actuator in the direction of fluid actuation, wherein the direction of fluid actuation corresponds to at least one of actuating the fluid actuator to inject fluid from a fluid reservoir and actuating the fluid actuator to draw fluid into the fluid reservoir.

Clause 2. The fluid injector system of clause 1, wherein the fluid actuator is at least one of a piston actuator, and pump actuator, and a compressive actuator.

Clause 3. The fluid injector system of clause 1 or 2, wherein the at least one processor is further programmed or configured to: determine a change in the orientation of the housing; and change the direction of fluid actuation in response to determining the change in the orientation of the housing.

Clause 4. The fluid injector system of any of clauses 1 to 3, wherein the orientation of the housing comprises a degree of tilt relative to a neutral plane.

Clause 5. The fluid injector system of any of clauses 1 to 4, wherein the at least one signal from the user input device comprises a rotation direction of the user input device.

Clause 6. The fluid injector system of any of clauses 1 to 5, wherein the at least one processor is further programmed or configured to: determine a load on the fluid actuator; and adjust a resistance of the user input device based on the load.

Clause 7. The fluid injector system of any of clauses 1 to 6, wherein the at least one processor is further programmed or configured to: determine at least one characteristic of a fluid path set; and adjust at least one parameter of the injection protocol based on the at least one characteristic of the fluid path set.

Clause 8. The fluid injector system of any of clauses 1 to 7, wherein the at least one characteristic of the fluid path set comprises a compliance rating of the fluid path set or of the fluid reservoir.

Clause 9. The fluid injector system of any of clauses 1 to 8, further comprising a scanner configured to scan a tag of the fluid path set to determine the at least one characteristic of the fluid path set.

Clause 10. The fluid injector system of any of clauses 1 to 9, wherein the at least one processor is further programmed or configured to: determine a current status of the fluid injector system; and disable at least one direction of fluid actuator movement based on the current status.

Clause 11. The fluid injector system of any of clauses 1 to 10, wherein the at least one processor is further programmed or configured to: set a fluid actuation speed based on at least one of: the at least one signal from the user input device; and a current status of the fluid injector system; and actuate the fluid actuator at the fluid actuation speed.

Clause 12. The fluid injector system of any of clauses 1 to 11, wherein the fluid actuation speed is set proportional to a speed at which the user input device is moved.

Clause 13. The fluid injector system of any of clauses 1 to 12, wherein the at least one processor is further programmed or configured to: receive at least one additional signal from the user input device; and adjust at least one of a height and the orientation of the housing based on the at least one additional signal.

Clause 14. The fluid injector system of any of clauses 1 to 13, further comprising at least one valve, wherein the at least one processor is further programmed or configured to actuate the valve in response to determining the direction of fluid actuation.

Clause 15. The fluid injector system of any of clauses 1 to 14, wherein the user input device is at least one of mounted to the housing and mounted remotely from the housing.

Clause 16. A computer program product for actuating a fluid actuator of a fluid injector system configured to perform an injection protocol, the computer program product comprising at least one non-transitory computer-readable medium comprising one or more instructions that, when executed by at least one processor, cause the at least one processor to: determine an orientation of a housing of the fluid injector system; receive at least one signal from a user input device of the fluid injector system; determine a direction of fluid actuation based on the orientation of the housing and the at least one signal; and actuate the fluid actuator in the direction of fluid actuation, wherein the direction of fluid actuation corresponds to at least one of actuating the fluid actuator to inject fluid from a fluid reservoir and actuating the fluid actuator to draw fluid into the fluid reservoir.

Clause 17. The computer program product of clause 16, wherein the fluid actuator is at least one of a piston actuator, and pump actuator, and a compressive actuator.

Clause 18. The computer program product of clause 16 or 17, wherein the one or more instructions further cause the at least one processor to: determine a change in the orientation of the housing; and change the direction of fluid actuation in response to determining the change in the orientation of the housing.

Clause 19. The computer program product of any of clauses 16 to 18, wherein the orientation of the housing comprises a degree of tilt relative to a neutral plane.

Clause 20. The computer program product of any of clauses 16 to 19, wherein the at least one signal from the user input device comprises a rotation direction of the user input device.

Clause 21. The computer program product of any of clauses 16 to 20, wherein the one or more instructions further cause the at least one processor to: determine a load on the fluid actuator; and adjust a resistance of the user input device based on the load.

Clause 22. The computer program product of any of clauses 16 to 21, wherein the one or more instructions further cause the at least one processor to: determine at least one characteristic of a fluid path set; and adjust at least one parameter of the injection protocol based on the at least one characteristic of the fluid path set.

Clause 23. The computer program product of any of clauses 16 to 22, wherein the at least one characteristic of the fluid path set comprises a compliance rating of the fluid path set or of the fluid reservoir.

Clause 24. The computer program product of any of clauses 16 to 23, wherein determining the at least one characteristic of a fluid path set comprises scanning a tag of the fluid path set.

Clause 25. The computer program product of any of clauses 16 to 24, wherein the one or more instructions further cause the at least one processor to: determine a current status of the fluid injector system; and disable at least one direction of fluid movement based on the current status.

Clause 26. The computer program product of any of clauses 16 to 25, wherein the one or more instructions further cause the at least one processor to: set a fluid actuation speed based on at least one of: the at least one signal from the user input device; and a current status of the fluid injector system; and actuate the fluid actuator at the actuation speed.

Clause 27. The computer program product of any of clauses 16 to 26, wherein the fluid actuation speed is set proportional to a speed at which the user input device is moved.

Clause 28. The computer program product of any of clauses 16 to 27, wherein the one or more instructions further cause the at least one processor to: receive at least one additional signal from the user input device; and adjust at least one of a height and the orientation of the housing based on the at least one additional signal.

Clause 29. The computer program product of any of clauses 16 to 28, wherein the one or more instructions further cause the at least one processor to: actuate at least one valve of the fluid injector system in response to determining the direction of fluid actuation.

Clause 30. A method for actuating a fluid actuator of a fluid injector system configured to perform an injection protocol, the method comprising: determining an orientation of a housing of the fluid injector system; receiving at least one signal from a user input device of the fluid injector system; determining a direction of fluid actuation based on the orientation of the housing and the at least one signal; and actuating the fluid actuator in the direction of fluid actuation, wherein the direction of fluid actuation corresponds to at least one of actuating the fluid actuator to inject fluid from a fluid reservoir and actuating the fluid actuator to draw fluid into the fluid reservoir.

Clause 31. The method of clause 30, wherein the fluid actuator is at least one of a piston actuator, and pump actuator, and a compressive actuator.

Clause 32. The method of clause 30 or 31, further comprising: determining a change in the orientation of the housing; and changing the direction of fluid actuation in response to determining the change in the orientation of the housing.

Clause 33. The method of any of clauses 30 to 32, wherein the orientation of the housing comprises a degree of tilt relative to a neutral plane.

Clause 34. The method of any of clauses 30 to 33, wherein the at least one signal from the user input device comprises a rotation direction of the user input device.

Clause 35. The method of any of clauses 30 to 34, further comprising: determining a load on the fluid actuator; and adjusting a resistance of the user input device based on the load.

Clause 36. The method of any of clauses 30 to 35, further comprising: determining at least one characteristic of a fluid path set; and adjusting at least one parameter of the injection protocol based on the at least one characteristic of the fluid path set.

Clause 37. The method of any of clauses 30 to 36, wherein the at least one characteristic of the fluid path set comprises a compliance rating of the fluid path set or of the fluid reservoir.

Clause 38. The method of any of clauses 30 to 37, wherein determining the at least one characteristic of a fluid path set comprises scanning a tag of the fluid path set.

Clause 39. The method of any of clauses 30 to 38, further comprising: determining a current status of the fluid injector system; and disabling at least one direction of fluid movement based on the current status.

Clause 40. The method of any of clauses 30 to 39, further comprising: setting a fluid actuation speed based on at least one of: the at least one signal from the user input device; and a current status of the fluid injector system; and actuating the fluid actuator at the actuation speed.

Clause 41. The method of any of clauses 30 to 40, wherein the fluid actuation speed is set proportional to a speed at which the user input device is moved.

Clause 42. The method of any of clauses 30 to 41, further comprising: receiving at least one additional signal from the user input device; and adjusting at least one of a height and the orientation of the housing based on the at least one additional signal.

Clause 43. The method of any of clauses 30 to 42, further comprising: actuating at least one valve of the fluid injector system in response to determining the direction of fluid actuation.

Further details and advantages of the various examples described in detail herein will become clear upon reviewing the following detailed description of the various examples in conjunction with the accompanying drawing figures.

### BRIEF DESCRIPTION OF THE DRAWINGS

FIG. 1A is a perspective view of a dual head fluid injector system according to an embodiment of the present disclosure;

FIG. 1B is a top view of the fluid injector system of FIG. 1A showing a user input device associated with each drive mechanism;

FIG. 2 is a schematic diagram of the fluid injector system of FIGS. 1A and 1B;

FIG. 3 is a schematic diagram of a rear view of the fluid injector system of FIGS. 1A and 1B;

FIG. 4 is a partial schematic diagram of an electronic controller of the fluid injector system of FIGS. 1A and 1B;

FIG. 5 is a partial schematic diagram of the electronic controller of the fluid injector system of FIGS. 1A and 1B; and

FIG. 6 is a flow diagram of a method of actuating a piston actuator according to an embodiment of the present disclosure.

### DETAILED DESCRIPTION OF THE DISCLOSURE

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to a syringe of a single or multi-patient disposable set, the term “proximal” refers to a portion of a syringe nearest a piston for delivering fluid from a syringe.

Spatial or directional terms, such as “left”, “right”, “inner”, “outer”, “above”, “below”, and the like, are not to be considered as limiting as the disclosure can assume various alternative orientations.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a fluid reservoir, such as a syringe, a rolling diaphragm, or multiple syringe disposable set, the term “distal” refers to a portion of the fluid reservoir nearest to a patient. When used in relation to a fluid reservoir, such as a syringe, a rolling diaphragm, or multiple syringe disposable set, the term “proximal” refers to a portion of the fluid reservoir nearest to the injector system.

Although the present disclosure is described primarily in the context of the MEDRAD® Stellant FLEX CT Injection System, it will be apparent to persons of ordinary skill in the art that the present disclosure can be applied to a variety of injection systems inclusive of their associated disposables (e.g., syringes, tubing, etc.). Examples of such injection systems include the MEDRAD® Stellant CT Injection System, the MEDRAD® Centargo CT Injection System, the MEDRAD® MRXperion MR Injection System, the MEDRAD® Mark 7 Arterion Injection System offered by Bayer HealthCare LLC, and other commercially available single-head and multi-head injection systems.

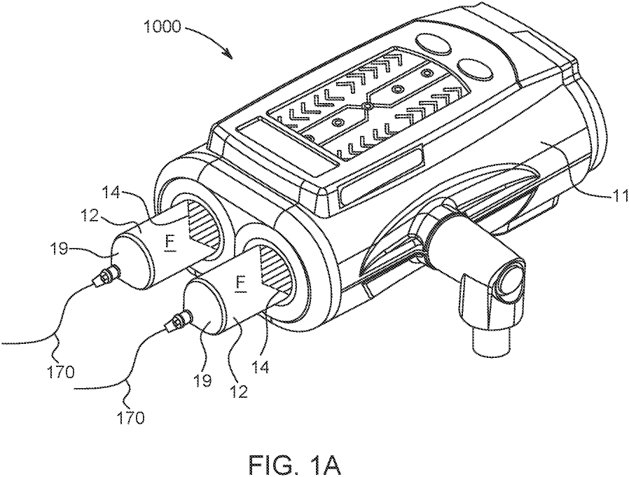

Referring now to the drawings in which like reference characters refer to like parts throughout the several views thereof, the present disclosure is generally directed to fluid injector systems, methods for operating fluid injector systems, and computer program products for executing such methods. Referring first to FIGS. 1A, 1B, 2 and 3, examples of a fluid injector system 1000 in accordance with the present disclosure include a housing 11 and at least one fluid reservoir, such as at least one syringe 12 or a fluid pump (not shown). The fluid injector system 1000 further includes a drive component to control fluid flow into or out of a fluid reservoir, such as a piston 13 associated with each of the syringes 12 that drives a plunger 14 within a barrel of the syringe 12. Each piston 13 may be independently driven by an associated fluid actuator 16, such as a linear actuator, ball screw, lead screw, rack-and-pinion, pump roller, or the like.

The description of the fluid injector system 1000 herein is generally directed to embodiments in which the fluid reservoir is a syringe 12 and the drive component for controlling fluid flow includes a piston 13 and plunger 14 operatively associated with the syringe 12. However, the present disclosure is to be understood as not limited to such embodiments. In particular, other embodiments of the system 1000 considered and encompassed by the present disclosure include a fluid pump as the fluid reservoir and a pump roller as the drive component. Other embodiments of the system 1000 considered and encompassed by the present disclosure include a bag as the fluid reservoir and a compressive actuator configured to compress the bag as the drive component. As such, reference in this description to “syringe” is to be understood to encompass any type of fluid reservoir including syringes, fluid pumps, bags, and the like. Reference in this description to “piston”, “plunger”, and “piston actuator” are likewise understood to encompass any device operatively associated with fluid reservoir and configured for controlling fluid flow into and out of the fluid reservoir. In particular, the term “fluid actuator” may be used herein to encompass a device or devices operatively associated with fluid reservoir and configured for controlling fluid flow into and out of the fluid reservoir. Particular examples of a “fluid actuator” as used herein includes the piston actuator 16 configured to actuate the piston 13 by extending and retracting the piston 13 within the syringe 12, a pump roller configured to actuate a fluid pump (e.g. a peristaltic pump) by compressing a tube associated with the fluid pump, and a compressive actuator configured to compress and/or squeeze a bag.

The fluid injector system 1000 is generally configured to deliver at least one medical fluid F, such as an imaging contrast media, saline solution, or any desired medical fluid, to a patient during an injection procedure. The at least one syringe 12 of the fluid injector system 1000 is configured to be filled with the at least one medical fluid F. Each syringe 12 may be filled with a different medical fluid F. The fluid injector system 1000 may be a multi-syringe injector, as shown, wherein several syringes 12 may be oriented side-by-side or in another spatial relationship and are separately actuated by respective pistons associated with the injector system 1000.

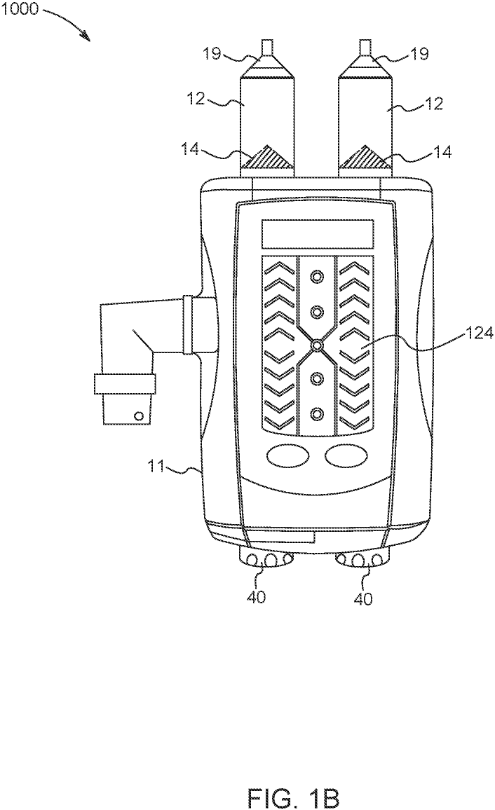

With continued reference to FIGS. 1A, 1B and 2, the fluid injector system 1000 may be used during a medical procedure to inject the at least one medical fluid F into the vasculature of a patient by driving the plungers 14 associated with the at least one syringe 12 with the at least one piston 13. The pistons 13 may be reciprocally operable upon the plungers 14. Upon engagement, the at least one piston 13 may move (retract) the plunger 14 toward a proximal end of the at least one syringe 12 to draw the medical fluid F into the at least one syringe 12 from a bulk fluid reservoir 120, such as a vial, bottle, or intravenous bag. The at least one piston 13 may further move (extend or push) the plungers 14 toward a distal end of the at least one syringe 12 to expel the medical fluid F from the at least one syringe 12 during a priming or fluid delivery step. The fluid injector system 1000 may further include a fluid path set 170 having at least one tube or tube set configured for fluid communication with each syringe 12 to place the syringes 12 in fluid communication with an administration line 176. A distal end of the administration line 176 may be configured for fluid communication with a catheter 178 inserted into a patient at a vascular access site. As such, fluid communication may be established between the syringes 12 and the patient such that the at least one medical fluid F can be injected from the syringes 12 into the patient.

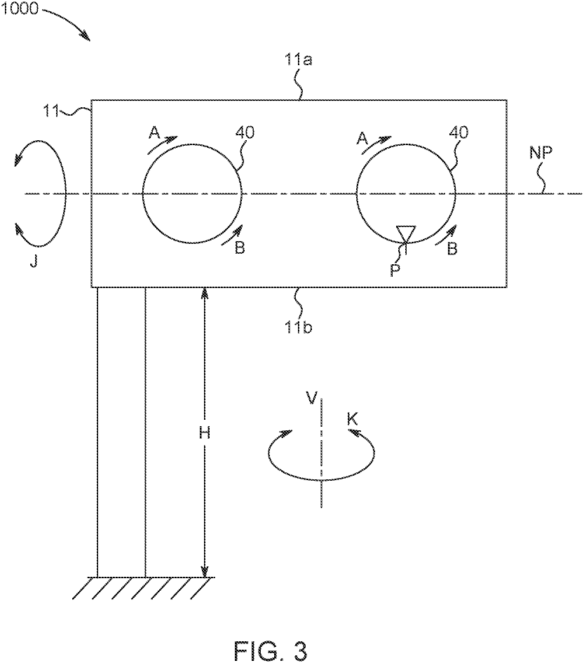

With continued reference to FIG. 2, the fluid injector system 1000 may further include at least one electronic controller 900 for controlling actuation of the at least one piston 13 via the piston actuators 16, and for controlling other components of the fluid injector system 1000. In some embodiments, the at least one controller 900 may be contained within the housing 11. In some embodiments, the at least one electronic controller 900 may be remotely mounted from the housing 11, such as in a separate room from the housing 11 so that the operator is not exposed to radiation during performance of a diagnostic procedure. In some embodiments, the at least one electronic controller 900 may include multiple components (as described herein with reference to FIG. 4, for example), of which some components are contained within the housing 11 and some components are remotely mounted from the housing 11.

The fluid injector system 1000 may be configured to perform one or more injection procedures according to one or more injection protocols stored in a memory accessible by the at least one controller 900. Prior to performing an injection procedure, however, air must be evacuated or purged from the syringe 12 prior to connecting the fluid path set 170 to the syringes 12. During the purging operation, the pistons 13 may be extended to a distalmost position in the corresponding syringes 12 so that air is forced out of the syringes 12. The syringes 12 and other portions of the fluid path 170 must then be filled. During the filling operation, the fluid path set 170 is connected to the syringes 12 and the pistons 13 may be retracted proximally to draw medical fluid F from the bulk fluid sources 120 into the syringes 12. The syringes 12 and other portions of the fluid path must then be primed. During the priming operation, the fluid injector system 1000 is typically oriented with its head (within housing 11) facing upward, allowing any air to accumulate at the tip of syringes 12. The pistons 13 may then be extended distally to push against plungers 14 to remove air from the syringes 12. The fluid path set 170 and the administration line 176 must also be primed according to known practice. Once the purging, filling and priming operations are complete, the administration line 176 may be connected to the catheter 178 inserted into the patient, and the pistons 13 may be extended distally in accordance with the injection protocol to inject medical fluid F from the syringes 12 into the patient.

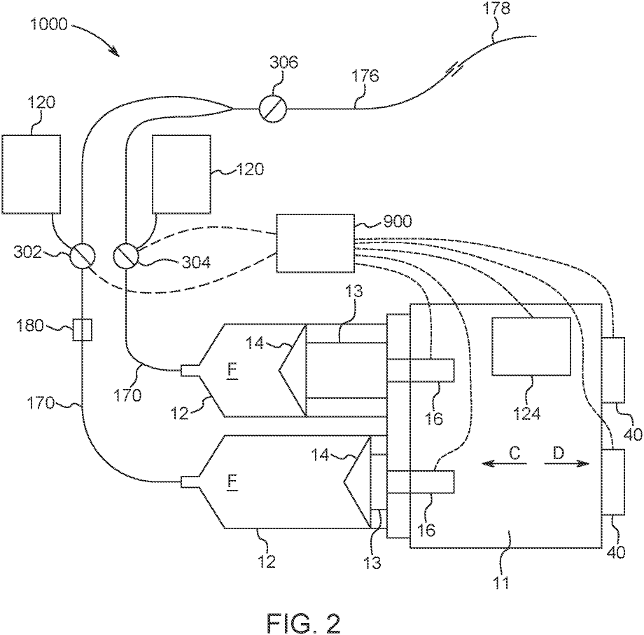

With continued reference to FIG. 2 and further reference to FIG. 3, the fluid injector system 1000 may include one or more user input devices 40 configured to allow manual control of the pistons 13 associated with the syringes 12. In some embodiments, each user input device 40 may include a rotatable knob, a rotatable dial, a lever, a slider, or another electromechanical element. In some embodiments, each user input device 40 may include a touchscreen. In some embodiments, each user input device 40 may include a microphone configured to receive voice commands from the operator. The user input devices 40 may be referred to hereinafter as “knobs 40” to avoid confusion with other components of the fluid injector system 1000. However, it is to be understood that all references herein to “knob 40” and “knobs 40” are not limiting and include all the other embodiments of the user input devices 40 described herein. In some embodiments, each knob 40 may be associated with one of the syringes 12. Each knob 40 may be in electrical communication with the at least one controller 900, such that, upon receiving at least one signal from the knob 40, the at least one controller 900 actuates the associated piston 13 via the associated actuator 16. For example, each knob 40 may be rotated in a first knob direction A (e.g. clockwise) to advance the piston 13 distally in a first piston direction C, and each knob 40 may be rotated in a second knob direction B (e.g. counter-clockwise) to retract the piston 13 proximally in a second piston direction D. The operator may, for example, rotate each knob 40 in the first direction A to advance the piston 13 distally in order to expel air bubbles from the fluid path set 170 during the purging operation as described above. Further, once the fluid path set 170 and the administration line 176 are connected to the syringes 12 and primed, the operator may rotate the knob 40 in the first direction A until medical fluid F is expelled from the administration line 176 so that a wet-to-wet connection is created when the administration line 176 is connected to the catheter 178. After the fluid path set 170 including the administration line 176 is primed and connected to the catheter 178, the operator may rotate the knobs 40 in the direction A to manually inject medical fluid F into the patient. Furthermore, the operator may rotate each knob 40 in the second direction B to retract the piston 13 proximally in order to draw fluid into the syringe 12 from the bulk fluid source 120.

In some embodiments, as shown in FIGS. 2 and 3, the knobs 40 may be mounted or embedded at any location on the housing 11 such as the back, side, top of the housing 11. In some embodiments, the knobs 40 may be remotely mounted from the housing 11, such as in a separate room from the housing 11 so that the operator may control the pistons 13 from a separate room not exposed to radiation during performance of a diagnostic procedure. In some embodiments, the knobs 40 may be mounted or embedded in a scanner (e.g. a CT, CV, PET, or MRI imaging device) configured for performing a diagnostic imaging procedure on the patient.

With continued reference to FIG. 2, the fluid injector system 1000 may include one or more user interfaces 124, such as a graphical user interface (GUI) display window. The user interface 124 may display information pertinent to a fluid injection procedure involving the fluid injector system 1000, such as injection status or progress, current flow rate, fluid pressure, and volume remaining in syringes 12 and in the at least one bulk fluid source 120 connected to the fluid injector system 1000. The interface 124 may be in electronic communication with the at least one controller 900 to allow a user to input parameters and control the processes of a fluid injection procedure. The user interface 124 may include one or more of touch screens, buttons, knobs, dials, sliders, microphones and the like that allow an operator to input commands and/or data for operation of the fluid injector system 1000.

With continued reference to FIG. 2, the fluid injector system 1000 may further include one or more valves 302, 304, 306 disposed at various locations along the fluid path set 170. Each of the valves 302, 304, 306 may be in the form of a shut-off valve and/or a flow rate control valve to regulate flow of the medical fluid F to the patient. Each of the valves 302, 304, 306 may be, for example, a stopcock, pinch valve, duckbill valve, or the like. In the embodiment shown in FIG. 2, the valves 302 and 304 are provided on the fluid path set 170 between the syringes 12 and the bulk fluid sources 120. The valve 306 is provided on the fluid path set 170 downstream of the valves 302 and 304.

Each of the valves 302, 304, 306 may be controllable by the at least one controller 900 to regulate the flow of the fluid F through the fluid path set 170. For example, any or all of the valves 302, 304, 306 may be closed by the controller 900 in response to detection of air in the fluid path set 170. During the fill operation, the valves 302 and 304 may be actuated by the controller 900 to provide fluid communication between the syringes 12 and the bulk fluid sources 120, such that the syringes 12 can draw medical fluid F from the bulk fluid sources 120. The valves 302 and 304 may isolate the syringes 12 and bulk fluid sources 120 from the administration line 176 during the fill phase to prevent the syringes 12 from drawing in fluid and/or air from the atmosphere. During the priming operation and the injection procedure itself, the valves 302 and 304 may be actuated to provide fluid communication between the syringes 12 and the administration line 176 to allow medical fluid F to be injected from the syringes 12 to the administration line 176. The valves 302 and 304 may isolate the bulk fluid sources 120 from the syringes 12 and the administration line 176 during the priming operation, and the injection procedure, so that medical fluid F cannot be injected into the bulk fluid sources 120. The valve 302 and 304 may also be selectively closed by the controller 900 to prevent backflow of pressurized medical fluid F from the fluid path set 170 into the syringes 12 due to a difference in pressure and/or fluid viscosity between the syringes 12 and fluid path set 170.

Further details and examples of suitable non-limiting powered injector systems, including syringes, controllers, air detectors, and/or fluid path sets are described in U.S. Pat. Nos. 5,383,858; 7,553,294; 7,666,169; 8,945,051; 10,022,493; and 10,507,319, the disclosures of which are hereby incorporated by reference in their entireties.

With continued reference to FIG. 3, the housing 11 may be repositioned in space by raising/lowering the housing 11 in a linear direction H, rotating the housing 11 relative to a neutral plane NP in a rotational direction J, and swiveling the housing 11 about a vertical axis V in a rotational direction K. In FIG. 3, the housing 11 is shown in a position in which a topside 11a of the housing 11 is oriented above an underside 11b. The housing 11 may be rotated and/or swiveled between a plurality of discrete, predetermined orientations, or between an infinite number of positions, to allow optimal positioning of the fluid injector system 1000 relative to the patient, operator, patient bed, and other objects in the scan room. For example, the housing 11 may be rotated approximately 180° in the direction J about the neutral plane NP from the position shown in FIG. 3 such that the underside 11b of the housing 11 is oriented above the topside 11a. Rotation of the housing 11 may affect the operator’s intuition as to what direction the knobs 40 must be rotated in order to drive the piston 13 in a desired direction. As such, embodiments of the present disclosure are directed to a method for correlating the rotation direction of the knobs 40 to the orientation of the housing 11.

With continued reference to FIG. 3, in some embodiments, the knobs 40 may be continuously rotatable, i.e. freewheeling, such that the rotation of each knob 40 causes movement of the corresponding piston 13 so long as the knob 40 is being continuously rotated. If the operator stops rotating the knob 40, movement of the corresponding piston 13 halts. The speed of movement of the piston 13 may be proportional to the speed at which the knob 40 is rotated in the direction A or B, as will be discussed in greater detail herein. In some embodiments, each knob 40 may be biased towards a neutral position P in which movement of the corresponding piston 13 halts. The knob 40 may be biased towards the neutral position P by a spring or like component. The speed of movement of the piston 13 may be proportional to the degree to which the knob 40 is rotated away from the neutral position P in the direction A or B.

Referring now to FIG. 4, a diagram of example components of the at least one electronic controller 900 for implementing and performing the systems and methods described herein is shown according to embodiments of the present disclosure. In some embodiments, the electronic controller 900 may include additional components, fewer components, different components, or differently arranged components than those shown in FIG. 4. The electronic controller 900 may include a bus 902, at least one processor 904, memory 906, a storage component 908, an input component 910, an output component 912, and a communication interface 914 (such as a GUI or other user interface). The bus 902 may include a component that permits communication among the components of the electronic controller 900. In some non-limiting embodiments, the at least one processor 904 may be implemented in hardware, firmware, or a combination of hardware and software. For example, the at least one processor 904 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 906 may include random access memory (RAM), read only memory (ROM), and/or another type of dynamic or static storage device (e.g., flash memory, magnetic memory, optical memory, etc.) that stores information and/or instructions for use by the at least one processor 904.

With continued reference to FIG. 4, the storage component 908 may store information and/or software related to the operation and use of the electronic controller 900. For example, the storage component 908 may include a hard disk (e.g., a magnetic disk, an optical disk, a magneto-optic disk, a solid state disk, etc.) and/or another type of computer-readable medium. The input component 910 may include a component that permits the electronic controller 900 to receive information, such as via user input (e.g., the GUI, a touch screen display, a keyboard, a keypad, a mouse, a button, a switch, a microphone, etc.). Additionally, or alternatively, the input component 910 may include a sensor for sensing information (e.g., a global positioning system (GPS) component, an accelerometer, a gyroscope, a scanner, etc.). The output component 912 may include a component that provides output information and/or commands from the electronic controller 900 (e.g., the GUI, a display, a speaker, one or more light-emitting diodes (LEDs), motors, actuators, solenoids, etc.). The communication interface 914 may include a transceiver-like component (e.g., a transceiver, a separate receiver and transmitter, etc.) that enables the electronic controller 900 to communicate with other devices, such as via a wired connection, a wireless connection, or a combination of wired and wireless connections. The communication interface 914 may permit the electronic controller 900 to receive information from another device and/or provide information to another device. For example, the communication interface 914 may include an Ethernet interface, an optical interface, a coaxial interface, an infrared interface, a radio frequency (RF) interface, a universal serial bus (USB) interface, a Wi-Fi® interface, a cellular network interface, Bluetooth, and/or the like. The input component 910, output component 912, and/or the communication interface 914 may correspond to, or be components of, the one or more user interface 124 (see FIG. 2).

With continued reference to FIG. 4, the electronic controller 900 may perform methods described herein based on the at least one processor 904 executing software instructions stored by a computer-readable medium, such as the memory 906 and/or the storage component 908. A computer-readable medium may include any non-transitory memory device. A memory device includes memory space located inside of a single physical storage device or memory space spread across multiple physical storage devices. Software instructions may be read into the memory 906 and/or the storage component 908 from another computer-readable medium or from another device via communication interface 914. When executed, software instructions stored in the memory 906 and/or storage component 908 may cause processor 904 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “programmed or configured,” as used herein, refers to an arrangement of software, hardware circuitry, or any combination thereof on one or more devices.

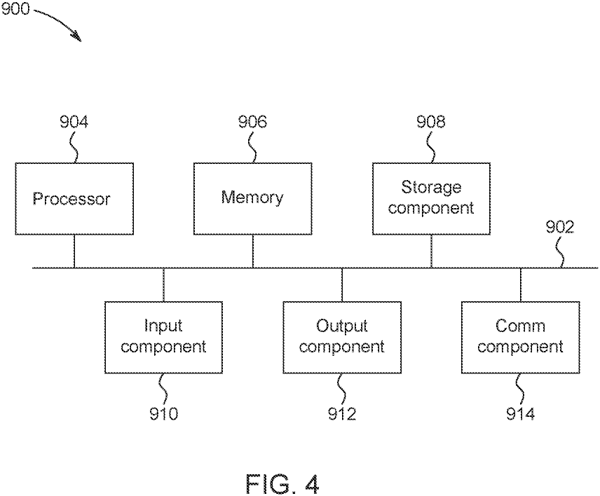

Referring now to FIG. 5, a schematic diagram of input components 910 and output components 912 of the electronic controller 900 is shown in accordance with embodiments of the present disclosure. The input components 910 may include the knobs 40 and the user interface 124. In some embodiments, the input components may further include at least one of a tilt sensor 802, a scanner 804, a pressure sensor 806, and a piston load sensor 808. The tilt sensor 802 may be configured to determine a position and/or orientation of the housing 11 of the fluid injector system 1000. The tilt sensor 802 may include, for example, a gyroscope, an accelerometer, or the like. The scanner 804 may be configured to identify a tag, such as a barcode, QR code, RFID tag, or the like. For example, the scanner 804 may be configured to identify a tag 180 (see FIG. 2) provided on the fluid path set 170, the administration line 176, and/or the syringe 12. The pressure sensor 806 may be configured to measure a fluid pressure and may be mounted at any location along the fluid path set 170 to measure a fluid pressure at that location. The piston load sensor 808 may be configured to measure a load on the piston 13, for example the load on the piston 13 due to fluid pressure. In some embodiments, the piston load sensor 808 may include an ammeter configured to measure current draw of the piston actuator 16, from which the load on the piston 13 can be determined.

The output component 912 may include the piston actuators 16 and the user interface 124. In some embodiments, the output components may further include at least one of a haptic feedback component 810, a housing actuator 812, and a valve actuator 814. The haptic feedback component 810 may be configured to alter the force required for the user to actuate the knob 40. In some embodiments, the haptic feedback component 810 may include an adjustable brake mechanically and/or electrically coupled to the knob 40. The housing actuator 812 may be configured to swivel, rotate, raise, and/or lower the housing 11. In some embodiments, the housing actuator 812 may include a motor, solenoid, linear actuator, or other electromotive component. The valve actuator 814 may be configured to actuate one or more of the valves 302, 304, 306 (see FIG. 2) and may include a motor, solenoid, linear actuator, or other electromotive component.

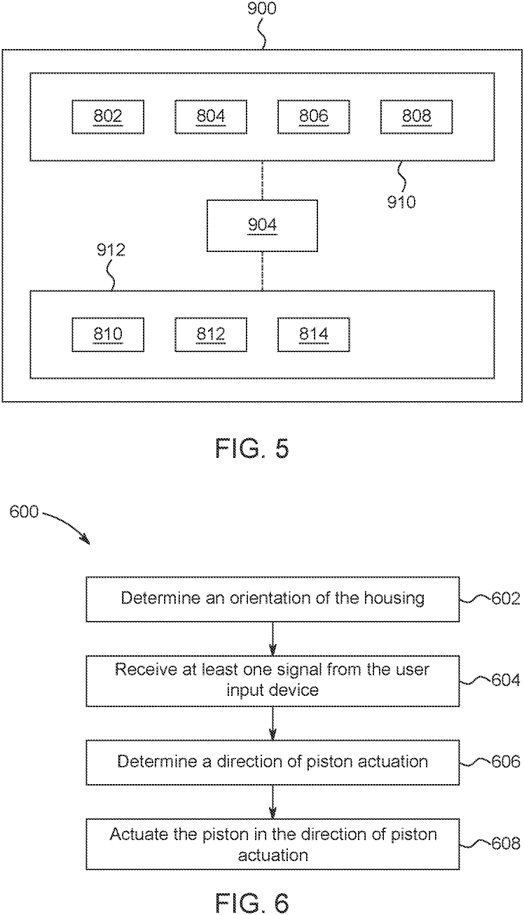

Referring now to FIG. 6, a flow diagram for a method 600 of actuating one or more pistons 13 of the fluid injector system 1000 is shown. Each step of the method 600 may be performed by the controller 900, more particularly by the at least one processor 904, of the fluid delivery system 1000. At step 602, the method may include determining an orientation of the housing 11 of the fluid injector system 1000. The at least one processor 904 may determine the orientation of the housing 11 via the tilt sensor 802. In particular, the at least one processor 904 may determine the orientation of the housing 11 relative to the neutral plane NP (see FIG. 3). The at least one processor 904 may particularly determine whether a topside 11a of the housing 11 is oriented above an underside 11b, as shown in FIG. 3, or whether the underside 11b of the housing 11 is oriented above the topside 11a, i.e. the opposite orientation shown in FIG. 3. In some embodiments, the at least one processor 904 may determine a degree of tilt of the housing 11 relative to the neutral plane NP.

At step 604, the method 600 may include receiving at least one signal from the user input device 40. The at least one signal may include a direction in which the user input device 40 is moved. For example, the at least one signal may include that the user input device 40 is moved (e.g. rotated) in the first direction A or the second direction B, as shown in FIG. 3. The at least one signal may further include a speed at which the user input device 40 is moved, or a degree to which the user input device 40 is moved. For example, the at least one signal may include the speed at which the user input device 40 is moved (e.g. rotated) in the first direction A or the second direction B. The at least one processor 904 may be programmed or configured to receive the at least one signal from the user input device 40.

At step 606, the method 600 may include determining a direction of fluid actuation based on the orientation of the housing 11 determined at step 602 and the at least one signal received at step 604. The direction of fluid actuation may correspond to at least one of actuating the fluid actuator to inject fluid from a fluid reservoir and actuating the fluid actuator to draw fluid into the fluid reservoir In some embodiments, the direction of fluid actuation corresponds to one of the piston directions C or D shown in FIG. 2. In a position of the housing 11 in which the topside 11a is oriented above the underside 11b (as shown in FIG. 3), the at least one processor 904 may be programmed or configured to move the piston 13 in the direction C when the user input device 40 is rotated in the direction A, and to move the piston 13 in the direction D when the user input device 40 is rotated in the direction B. Inversely, in a position of the housing 11 in which the underside 11b is oriented above the topside 11a (i.e. opposite the position shown in FIG. 3), the at least one processor 904 may be programmed or configured to move the piston 13 in the direction C when the user input device 40 is rotated in the direction B, and to move the piston 13 in the direction D when the user input device 40 is rotated in the direction A. Thus, if the housing 11 is oriented such that the topside 11a is above the underside 11b and the user input device 40 is rotated in the direction A, the at least one processor 904 may determine at step 606 that the direction of fluid actuation corresponds to the direction C. Alternatively, if the housing 11 is oriented such that the topside 11a is above the underside 11b and the user input device 40 is rotated in the direction B, the at least one processor 904 may determine at step 606 that the direction of fluid actuation corresponds to the direction D. Alternatively, if the housing 11 is oriented such that the underside 11b is above the topside 11a and the user input device 40 is rotated in the direction A, the at least one processor 904 may determine at step 606 that the direction of fluid actuation corresponds to the direction D. Alternatively, if the housing 11 is oriented such that the underside 11b is above the topside 11a and the user input device 40 is rotated in the direction B, the at least one processor 904 may determine at step 606 that the direction of fluid actuation corresponds to the direction C.

By changing the direction of fluid actuation relative to rotation of the user input device 40 when the orientation of the housing 11 is inverted, rotating the user input device 40 in one direction always results in the same direction of piston 13 movement regardless of the orientation of the housing 11. Thus, actuation of the user input device 40 may be intuitive for the operator because the operator does not have to consider the orientation of the housing 11 in determining which direction to move the user input device 40 to achieve a desired movement of the piston 13.

In some embodiments, the user interface 124 may display a graphic or message indicating the correlation between movement of the user input devices 40 and movement of the piston 13 to further assist the operator in moving the user input devices 40 in an intended direction.

At step 608, the method 600 may include actuating the piston 13 in the direction of fluid actuation determined at step 606. Thus, if the at least one processor 904 determines at step 606 that the direction of fluid actuation corresponds to the direction C, the at least one processor 904 may be programmed or configured to move the piston 13 in the direction C to advance the piston 13 distally within the syringe 12. If the at least one processor 904 determines at step 606 that the direction of fluid actuation corresponds to the direction D, the at least one processor 904 may be programmed or configured to move the piston 13 in the direction D to retract the piston 13 proximally within the syringe 12. As described herein with reference to FIGS. 1A, 1B, and 2, advancing the piston 13 in the direction C can be performed to purge air from the syringes 12. Further, advancing the piston 13 in the direction C can be performed to prime the fluid path set 170 and to create a fluid bubble at a distal end of the administration line 176 to form a wet-to-wet connection with the catheter 178. Retracting the piston 13 in the direction D proximally draws fluid F into the syringe 12, and can therefore be performed to fill the syringe 12 from the bulk fluid source 120.

In some embodiments, the method 600 may include determining a change in orientation of the housing 11. In particular, the at least one processor 904 may be programmed or configured to determine, via the tilt sensor 802, that the orientation of the housing 11 has been changed from the orientation determined at step 602. In response to determining the change in orientation of the housing 11, the at least one processor 904 may change the direction of fluid actuation that was determined at step 606. That is, if the at least one processor 904 determined at step 606 that the direction of fluid actuation corresponds to the direction C, the at least one processor 904 may change the direction of fluid actuation to correspond to the direction D (or vice versa) in response to determining the change in orientation of the housing 11. As such, changing the orientation of the housing 11 does not change the direction in which the operator must move (e.g. rotate) the user input device 40 to achieve the desired movement of the piston 13.

In some embodiments, the method 600 may include determining a load on the piston actuator 16 and adjusting a resistance of the user input device 40 based on the load. In particular, the at least on processor 904 may determine a load on the piston actuator 16 due to fluid pressure in the associated syringe 12 and/or the fluid path set 170. The fluid pressure may be measured directly by the pressure sensor 806, or may be determined from a current draw of the piston actuator 16 as measured by the piston load sensor 808. Based on the load on the piston actuator 16, the at least one processor 904 may be programmed or configured to adjust the resistance of the user input device 40 to increase or decrease the force required by the operator to move (e.g. rotate) the user input device 40. In some embodiments, the at least one processor 904 may increase the resistance of the user input device 40 as the load on the piston actuator 16 increases. Thus, the resistance applied to the user input device 40 by the at least one processor 904 mimics the resistance that would be felt by the operator if the user input device 40 was directly mechanically coupled to the piston actuator 16 (i.e. if the operator has to overcome the load acting through the piston actuator 16 in order to rotate the user input device 40). An increase in the resistance to movement of the user input device 40 may allow the operator to feel fluid pressure increases in the system 1000, such as fluid pressure increases caused by occlusions in the fluid path set 170 and/or the syringes 12. In some embodiments, the at least one processor 904 may be programmed or configured to adjust the resistance of the user input device 40 via the haptic feedback component 810.

In some embodiments, the method 600 may include determining at least one characteristic of the fluid path set 170, the administration line 176, and/or the syringe 12. In some embodiments, the at least one processor 904 may be programmed or configured to determine the at least one characteristic via the scanner 804 identifying the tag 180. In some embodiments, the at least one characteristic may include a volume, length, and/or pressure rating of the syringe 12, the fluid path set 170, and/or the administration line 176. In some embodiments, the at least one characteristic may include a type of fluid F prefilled into the syringe 12. In response to determining the at least one characteristic, the at least one processor 904 may be programmed or configured to adjust at least one parameter of the injection protocol. For example, the at least one processor 904 may be programmed or configured to adjust travel limits on the piston 13 based on the volume and/or length of the syringe 12. As such, the at least one processor 904 may limit the distance that the piston actuator 16 can move in the directions C and D. If the user input device 40 is rotated when the piston 13 is already at a travel limit, the at least one processor 904 prevents further actuation of the piston actuator 16 to prevent the piston 13 from traveling beyond the travel limit.

In another example, if the tag 180 on one (or more) of the fluid path set 170, the administration line 176, and/or the syringe 12 contains information indicating that the compliance rating thereof is different from a typical value, the at least one processor 904 may be programmed or configured to adjust, for example, the fluid actuation speed, and thus the flow rate, based on the compliance rating to improve accuracy of the injection procedure. For example, the at least one processor 904 may be programmed or configured to, upon receiving a command from the user input device 40 to extend the piston 14, automatically extend the piston 14 an additional predetermined distance to account for compliance and/or mechanical slack in the syringe 12, the fluid path set 170, and/or the administration line 176. The additional predetermined distance that the piston 14 is extended may be based on the compliance rating read from the tag 180.

In another example, the tag 180 on one (or more) of the fluid path set 170 and/or the syringe 12 may contain information indicating whether the fluid path set 170 and/or the syringe 12 is a single use component (i.e. only intended for use on a single patient and/or for a single injection procedure) or a multi-use component (i.e. intended for use on multiple patients and/or for multiple injection procedures). If the tag 180 indicates that the fluid path set 170 and/or the syringe 12 is a single use component, the at least one processor 904 may be programmed or configured to disable functions and/or features of the system 1000 if the fluid path set 170 and/or the syringe 12 is not replaced after an injection procedure. For example, the at least one processor 904 may be programmed or configured to override any command input by the operator via the knobs 40 if the fluid path set 170 and/or the syringe 12 is not replaced after an injection procedure.

In some embodiments, the method 600 may include determining a current status of fluid injection system 1000. The current status of the fluid injection system may be input into the controller 900 via the user interface 124 or may be automatically initiated by the controller 900. The statuses of the fluid injection system may include, for example, the purging operation, the filling operation, the priming operation, and the injection procedure. In response to determining the current status of the fluid injection system, the at least one processor 904 may be programmed or configured to disable piston movement in at least one direction. For example, during the filling operation, the at least one processor 904 may be configured to disable movement of the piston 13 in the direction C so that fluid is not inadvertently injected from the syringe 12 into the bulk fluid source 120. If the user input device 40 is moved (e.g. rotated) in a direction that would cause the piston 13 to move in the direction C, the at least one processor 904 may be programmed or configured to override the user input device 40 and not actuate the piston actuator 16. Likewise, during the priming operation or purging operation, the at least one processor 904 may be configured to disable movement of the piston 13 in the direction D so that air is not inadvertently drawn into the syringe 12, the fluid path set 170, and/or the administration line 176. If the user input device 40 is moved (e.g. rotated) in a direction that would cause the piston 13 to move in the direction D, the at least one processor 904 may be programmed or configured to override the user input device 40 and not actuate the piston actuator 16. Likewise, while an injection procedure is being performed according to the desired injection protocol, the at least one processor 904 may be configured to disable movement of the piston 13 in the direction D so that fluid is not inadvertently drawn out of the patient. If the user input device 40 is rotated in a direction that would cause the piston 13 to move in the direction D, the at least one processor 904 may be programmed or configured to override the user input device 40 and not actuate the piston actuator 16. Similarly, the at least one processor 904 may be configured not to alter or in any way affect or override the movement of piston 13 in the direction C, thus leaving the movement of piston 13 in the direction C solely under the control of fluid injection system 1000 according to the programmed injection protocol.

In some embodiments, the method 600 may include setting a fluid actuation speed based on the at least one signal from the user input device 40. As described herein with reference to step 604, the at least one signal from the user input device 40 may include the speed at which the user input device 40 is moved (e.g. rotated) in the first direction A or the second direction B. In some embodiments, the at least one processor 904 may be programmed or configured to set the fluid actuation speed to be proportional to the speed at which the user input device 40 is moved. This may mimic the behavior of a fluid injector system in which the knob is directly mechanically coupled to the piston actuator, such as the knob being directly mechanically coupled to a ball screw. In some embodiments, the at least one signal from the user input device 40 may include a degree to which the user input device 40 is moved from the neutral position P (see FIG. 3) during the purging operation, and the at least one processor 904 may be programmed or configured to set the fluid actuation speed to be proportional to the degree to which the user input device 40 is moved from the neutral position P. In some embodiments, the at least one processor 904 may be programmed or configured to set the fluid actuation speed to a constant, predetermined speed regardless of the speed at which the user input device 40 is moved or the degree to which the user input device 40 is moved from the neutral position P. The at least one processor 904 may be programmed or configured to actuate the piston actuator 16 at the determined fluid actuation speed at step 608.

In some embodiments, the method 600 may include setting the fluid actuation speed based on the current status of fluid injection system 1000. For example, the at least one processor 904 may be programmed or configured to set the fluid actuation speed to be a faster speed during the purging operation, during which air is evacuated from the syringe 12, than during the filling and priming operations and the injection procedure. As such, moving the user input device 40 at one speed during the purging operation will result in a faster fluid actuation speed than moving the user input device 40 at the same speed during the filling and priming operations, and the injection procedure. The at least one processor 904 may be programmed or configured to actuate the piston actuator 16 at the determined fluid actuation speed at step 608.

In some embodiments, the method 600 may include setting the fluid actuation speed based on a combination of the at least one signal from user input device 40 and the current status of fluid injection system 1000. For example, the at least one processor 904 may be programmed or configured to set the fluid actuation speed proportional to the speed at which the user input device 40 is moved, multiplied by an additional speed factor constant dependent upon the current status of the fluid injection system 1000. For example, the additional speed factor may be higher if the system 1000 is being purged than if the system 1000 is being filled or primed or being used to perform an injection procedure. The at least one processor 904 may be programmed or configured to actuate the piston actuator 16 at the determined fluid actuation speed at step 608.

In some embodiments, the method 600 may include receiving at least one additional signal from the user input device 40. The at least one additional signal may correspond to a command to adjust a position, orientation, or height of the housing 11. The at least one processor 904 may be programmed or configured to adjust at least one of the position, orientation, and height of the housing 11 based on the at least one additional signal. In some embodiments, the operator may input a command into the user interface 124 to enter one or more “housing adjustment modes” in which the user input device 40 is decoupled from the piston actuator 16 and instead coupled to the at least one housing actuator 812. When in the “housing adjustment modes”, movement of the user input device 40 in the directions A and B generates the at least one additional signal received by the at least one processor 904. The at least one processor 904 may actuate the at least one housing actuator 812 based on the at least one additional signal to raise, lower, rotate, and/or swivel the housing 11. The one or more “housing adjustment modes” may include, for example, a “raise/lower mode” in which movement of the user input device 40 raises and lowers the housing 11 in the direction H (see FIG. 2). For example, the at least one processor 904 may be programmed or configured such that movement of the user input device 40 in the direction A raises the housing 11 and movement of the user input device 40 in the direction B lowers the housing 11. The one or more “housing adjustment modes” may further include, for example, a “rotate mode” in which movement of the user input device 40 rotates the housing 11 in the direction J relative to the neutral plane NP. For example, the at least one processor 904 may be programmed or configured such that movement of the user input device 40 in the direction A rotates the housing 11 clockwise and movement of the user input device 40 in the direction B rotates the housing 11 counterclockwise. The one or more “housing adjustment modes” may further include, for example, a “swivel mode” in which movement of the user input device 40 rotates the housing 11 in the direction K relative to the vertical axis V. For example, the at least one processor 904 may be programmed or configured such that movement of the user input device 40 in the direction A rotates the housing 11 clockwise and movement of the user input device 40 in the direction B rotates the housing 11 counterclockwise. Once the housing is in the operator’s desired position, the operator may input a command into the user interface 124 to exit the “housing adjustment modes” and recouple the user input device 40 to the piston actuator 16.

In some embodiments, the method 600 may include actuating one or more of the valves 302, 304, 306 in response to determining the direction of fluid actuation at step 606. The at least one processor 904 may actuate the valve actuator 814 to open, close, or otherwise actuate one or more of the valves 302, 304, 306 to establish or break fluid communication between various components of the fluid injector system 1000. For example, if the direction of fluid actuation corresponds to the direction D, i.e. retraction of the piston, the at least one processor 904 may actuate the valve 302 to establish fluid communication between the syringe 12 and the bulk fluid source 120. Retraction of the piston 13 in the direction D will thus draw fluid into the syringe 12 from the bulk fluid source 120. The at least one processor 904 may also close the valve 306 to prevent fluid and/or air from being drawn into the syringe 12 from a distal end of the fluid path set 170 and/or the administration line 176.

Alternatively, if the direction of fluid actuation corresponds to the direction D, i.e. extension of the piston, the at least one processor 904 may actuate the valve 302 to isolate the bulk fluid source 120 to prevent injection of fluid F from the syringe 12 into the bulk fluid source 120.

In some embodiments, a default setting for the direction of fluid actuation may be customized by the operator. In particular, the operator may program the default setting for the direction of fluid actuation in the at least one processor 904 via the user interface 124. For example, the operator may set the default setting such that rotation of the user input device 40 in the direction A while the housing 11 is oriented as shown in FIG. 3 (i.e. the topside 11a is oriented above the underside 11b) causes the piston 13 to move in the direction C. Alternatively, the operator may change the default setting such that rotation of the user input device 40 in the direction A while the housing 11 is oriented as shown in FIG. 3 (i.e. the topside 11a is oriented above the underside 11b) causes the piston 13 to move in the direction D.

In some embodiments, the at least one processor 904 may be programmed or configured to determine a load applied to the piston 13 by fluid pressure of the system 1000. Because the piston actuator 16 is in electrical communication with the user input device 40 rather than mechanically coupled to the user input device 40, the at least one processor 904 can distinguish the load applied to the piston 13 by the operator from the load due to fluid pressure. In contrast, systems in which the knobs are directly mechanically connected to the piston actuators generally cannot differentiate between load applied by the operator and load due to fluid pressure.

In some embodiments, the present disclosure is directed to a computer program product for causing at least one processor to execute the method 600. In some embodiments, the present disclosure is directed to a fluid injector system having at least one processor configured to execute the method 600.

While examples of fluid injector systems, methods of operation thereof, and computer program products were provided in the foregoing description, those skilled in the art may make modifications and alterations to these examples without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

1. A fluid injector system configured to perform an injection protocol, the fluid injector system comprising:
a housing; and
a controller operatively associated with a user input device and a fluid actuator, the controller comprising at least one processor programmed or configured to:
determine an orientation of the housing;
receive at least one signal from the user input device;
determine a direction of fluid actuation based on the orientation of the housing and the at least one signal; and
actuate the fluid actuator in the direction of fluid actuation,
wherein the fluid actuator is at least one of a piston actuator, and pump actuator, and a compressive actuator; and
wherein the direction of fluid actuation corresponds to at least one of actuating the fluid actuator to inject fluid from a fluid reservoir and actuating the fluid actuator to draw fluid into the fluid reservoir.

2. (canceled)

3. The fluid injector system of claim 1, wherein the at least one processor is further programmed or configured to:
determine a change in the orientation of the housing; and
change the direction of fluid actuation in response to determining the change in the orientation of the housing;
wherein the orientation of the housing comprises a degree of tilt relative to a neutral plane.

4. (canceled)

5. The fluid injector system of claim 1, wherein the at least one signal from the user input device comprises a rotation direction of the user input device.

6. The fluid injector system of claim 1, wherein the at least one processor is further programmed or configured to:
determine a load on the fluid actuator; and
adjust a resistance of the user input device based on the load.

7. The fluid injector system of claim 1, wherein the at least one processor is further programmed or configured to:
determine at least one characteristic of a fluid path set; and
adjust at least one parameter of the injection protocol based on the at least one characteristic of the fluid path set;
wherein the at least one characteristic of the fluid path set comprises a compliance rating of the fluid path set or of the fluid reservoir.

8. (canceled)

9. The fluid injector system of claim 7, further comprising a scanner configured to scan a tag of the fluid path set to determine the at least one characteristic of the fluid path set.

10. The fluid injector system of claim 1, wherein the at least one processor is further programmed or configured to:
determine a current status of the fluid injector system; and
disable at least one direction of fluid actuator movement based on the current status.

11. The fluid injector system of claim 1, wherein the at least one processor is further programmed or configured to:
set a fluid actuation speed based on at least one of:
the at least one signal from the user input device; and
a current status of the fluid injector system; and
actuate the fluid actuator at the fluid actuation speed;
wherein the fluid actuation speed is set proportional to a speed at which the user input device is moved.

12. (canceled)

13. The fluid injector system of claim 1, wherein the at least one processor is further programmed or configured to:
receive at least one additional signal from the user input device; and
adjust at least one of a height and the orientation of the housing based on the at least one additional signal.

14. The fluid injector system of claim 1, further comprising at least one valve,
wherein the at least one processor is further programmed or configured to actuate the valve in response to determining the direction of fluid actuation.

15. The fluid injector system of claim 1, wherein the user input device is at least one of mounted to the housing and mounted remotely from the housing.

16. A computer program product for actuating a fluid actuator of a fluid injector system configured to perform an injection protocol, the computer program product comprising at least one non-transitory computer-readable medium comprising one or more instructions that, when executed by at least one processor, cause the at least one processor to:
determine an orientation of a housing of the fluid injector system;
receive at least one signal from a user input device of the fluid injector system;
determine a direction of fluid actuation based on the orientation of the housing and the at least one signal; and
actuate the fluid actuator in the direction of fluid actuation,
wherein the fluid actuator is at least one of a piston actuator, and pump actuator, and a compressive actuator; and
wherein the direction of fluid actuation corresponds to at least one of actuating the fluid actuator to inject fluid from a fluid reservoir and actuating the fluid actuator to draw fluid into the fluid reservoir.

17. (canceled)

18. The computer program product of claim 16, wherein the one or more instructions further cause the at least one processor to:
determine a change in the orientation of the housing; and
change the direction of fluid actuation in response to determining the change in the orientation of the housing;
wherein the orientation of the housing comprises a degree of tilt relative to a neutral plane.

19. (canceled)

20. The computer program product of claim 16, wherein the at least one signal from the user input device comprises a rotation direction of the user input device.

21. The computer program product of claim 16, wherein the one or more instructions further cause the at least one processor to:
determine a load on the fluid actuator; and
adjust a resistance of the user input device based on the load.

22. The computer program product of claim 16, wherein the one or more instructions further cause the at least one processor to:
determine at least one characteristic of a fluid path set; and
adjust at least one parameter of the injection protocol based on the at least one characteristic of the fluid path set;
wherein the at least one characteristic of the fluid path set comprises a compliance rating of the fluid path set or of the fluid reservoir.

23. (canceled)

24. The computer program product of claim 22, wherein determining the at least one characteristic of a fluid path set comprises scanning a tag of the fluid path set.

25-43. (canceled)

44. A fluid injector system configured to perform an injection protocol, the fluid injector system comprising:
a housing;
a fluid actuator operatively associated with a fluid reservoir and configured for controlling fluid flow into and out of the fluid reservoir;
a user input device mounted remotely from the housing and configured for controlling of the fluid actuator; and
a controller operatively associated with the user input device and the fluid actuator, the controller comprising at least one processor programmed or configured to:
receive at least one signal from the user input device;
determine a direction of fluid actuation based on at least one of an orientation of the housing and the at least one signal; and
actuate the fluid actuator in the direction of fluid actuation,
wherein the direction of fluid actuation corresponds to at least one of actuating the fluid actuator to inject fluid from the fluid reservoir and actuating the fluid actuator to draw fluid into the fluid reservoir.

45. The fluid injector system of claim 44, wherein the user input device includes at least one of a touchscreen, a slider, a button, a knob, a dial and a microphone.

46. The fluid injector system of claim 45, wherein the user input device is one of:
embedded in a scanner configured for performing a diagnostic imaging procedure on a patient; and
in a separate room from the housing.

47. The fluid injector system of claim 44, wherein the controller is remotely mounted from the housing.

48. The fluid injector system of claim 44, wherein the controller includes some components thereof that are remotely mounted from the housing.
